(19) United States
(12) Patent Application Publication
(10) Pub. No.: US 2008/0269772 A1
(43) Pub. Date: Oct. 30, 2008

(54) Apparatus for fixing myoma during performing laparoscopic myomectomy

(76) Inventor: Joong Sub Choi, Seongnam-si (KR)

Correspondence Address: BACON & THOMAS, PLLC, 625 SLATERS LANE, FOURTH FLOOR, ALEXANDRIA, VA, 22314-1176, US

(21) Appl. No.: 11/976,983

(22) Filed: Oct. 30, 2007

(30) Foreign Application Priority Data
Apr. 30, 2007 (KR) 10-2007-0042185

(51) Int. Cl. A61B 17/42

(52) U.S. Cl. 606/119

(57) ABSTRACT

A myoma screw, for firmly fixing a uterine myoma of a patient to extract the uterine myoma easily during a laparoscopic myomectomy, wherein the apparatus is introduced into an abdominal cavity of the patient through a cannula, includes: a body part having a cross-sectional diameter smaller than that of the cannula; a fixing part, having a shape of a circular cone, for fixing the uterine myoma firmly; and a gripping part for applying traction force to the fixing part fixed into the uterine myoma. By using the apparatus during the laparoscopic myomectomy, the uterine myoma can be extracted safely, while obtaining a sufficient view of an operative field in a restricted space created by the cannula.

### FIELD OF THE INVENTION

The present invention relates to an apparatus for fixing a myoma; and, more particularly, to an apparatus for firmly and deeply fixing a uterine myoma during a laparoscopic myomectomy, thereby facilitating the enucleation of the uterine myoma from normal uterine tissue.

### BACKGROUND OF THE INVENTION

In recent years, the age of first pregnancy has been delayed due to social and cultural changes as well as widespread use of contraceptives. A myoma of the uterus, which is common to women over 30 years of age, may cause problems during pregnancy.

One of methods for enucleating the uterine myoma of patients is a laparoscopic myomectomy (LM), which currently brings more attention because of increased demands on uterine conservation and less invasive management of the uterine myoma. The laparoscopic myomectomy is a surgical procedure which requires the steps of: making a plurality of incisions in the skin of an abdomen; and performing a surgical operation for the uterine myoma in the abdominal cavity by using a laparoscope, a thin fiber-optic scope, which may be introduced into the abdominal cavity through the incisions whose sizes are smaller than those of traditional surgical procedures.

In comparison with the traditional abdominal myomectomy, the laparoscopic myomectomy provides the advantages of shorter hospitalization, faster recovery, fewer adhesions, less blood loss, and better cosmetic effect.

For the purpose of removing the uterine myoma efficiently, there is a need of a myoma screw, capable of controlling the location of the uterine myoma while the myoma screw is firmly fixated thereinto, thereby providing a good surgical view with ease.

To be more specific, after a capsule of the uterine myoma is partially dissected, the myoma screw may be firmly inserted into the partially dissected portion of the capsule, which enables operators to dissect all the capsule with the aid of other surgical instruments such as grasping forcpes or surgical scissors, thereby separating the uterine myoma from the capsule efficiently.

FIG. 1 shows a general view of a conventional myoma screw 10 for fixing the uterine myoma in laparoscopic myomectomy.

Referring to FIG. 1, the myoma screw 10 includes a body part 11, a fixing part 12 and a gripping part 13, which was manufactured by Karl Storz company of Germany (see http://www.karlstorz.de/).

The body part 11, having a cylindrical shape with a length of about 36 cm, made of metal, may be introduced into an abdominal cavity following the fixing part 12 through a cannula (not shown) during the operations.

The fixing part 12, having a shape of a spring or a wine screw with a length of about 12 cm, made of the metal same as that of the body part 11. The fixing part 12 may be fixated into the uterine myoma in order to prevent the uterine myoma from being shaken during the operations for removing the uterine myoma.

The body part 11 is connected to the fixing part 12 through a connecting part “A” by way of welding therebetween, the connecting part “A” having a cross-sectional diameter of about 2.2 cm, which is much smaller than that of the body part 11.

The gripping part 13, having an almost round shape like a spoon with a length of about 8 cm, made of the same metallic object as the body part 11, is connected to the body part 11 by way of, e.g., the welding. An operator may control his or her fingers to rotate and push the gripping part 13 such that the fixing part 12 is inserted into the uterine myoma.

Then, the position of the gripping part 13 may be shifted by applying a force thereto in order to move the position of the uterine myoma, so that a better view for the uterine myoma can be acquired during the laparoscopic myomectomy. That is to say, the controllability of the uterine myoma is required to get rid of the uterine myoma correctly and precisely during the laparoscopic myomectomy when only limited space for observing the uterine myoma is permitted. As the effective enucleation of the uterine myoma from normal uterine tissue is the most crucial step during the laparoscopic myomectomy, the good surgical vision should be obtained to fully extract the uterine myoma effectively irrespective of its location and size.

Moreover, in case the size of the uterine myoma is large, the surgical vision cannot be obtained sufficiently and the uterine myoma is much more difficult to enucleate because the narrow space only is available inside the abdominal cavity. Further, it may be difficult to control the myoma screw 10 due to the large size of the uterine myoma during the laparoscopic myomectomy, e.g., hemostasis, uterine closure, tissue removal and the like.

Accordingly, there has been needed a myoma screw capable of firmly fixing the uterine myoma and sufficiently applying force to the uterine myoma during the traction thereof.

However, the conventional myoma screw 10 has caused some drawbacks during the laparoscopic myomectomy as follows:

First, since the myoma screw 10 has a shape of a spring or a wine screw, the myoma screw 10 cannot be firmly fixated to the uterine myoma. In particular, the softening of the large uterine myoma due to degenerative changes thereof makes it very difficult to exert sufficient force onto the fixing part 12, thus failing to perform maneuvers such as traction-coutertraction and insertion thereof.

Second, excessive load may be applied to the connecting part “A” between the body part 11 and the fixing part 12 while the large-sized uterine myoma is manipulated in all directions with a view to obtaining a better view of an operative field through small incisions prepared on a patient's abdomen, with the consequence that the connecting part “A” might be easily broken. Therefore, the broken fixing part 12 may be stuck into the uterine myoma, thereby paying more attention to remove the broken fixing part 12 from the uterine myoma than that to perform the myomectomy.

Third, since the gripping part 13 occupies a small area with a round shape, the operator might have the difficulty in handling the gripping part 13 when the operator wants to rotate and push the gripping part 13 in order to fix the fixing part 12 to the uterine myoma. In specific, excessive load may be delivered to the tips of the operator's thumb and index finger when the operator is willing to exert a force on the gripping part 13. Accordingly, it is difficult to exert the force on the gripping part 13 efficiently at the time when the operator rotates and moves the position of the gripping part 13 to apply the traction force on the uterine myoma. This could compel the operator to control his or her fingers with an excessive burden, resulting in an arthralgia at the fingers.

### SUMMARY OF THE INVENTION

It is, therefore, an object of the present invention to provide an apparatus for firmly and deeply fixing a uterine myoma, even in case the uterine myoma is softened due to degenerative changes, during a laparoscopic myomectomy.

It is another object of the present invention to provide an apparatus for applying a traction force, such as pushing and pulling force, on a large uterine myoma in all directions, while preventing a fixing part thereof from being easily separated from a body part thereof, thereby obtaining a magnified view of an operative field.

It is another object of the present invention to provide an apparatus for applying a traction force on the uterine myoma while controlling the gripping part thereof by the entire palm of the operator's hand at the time when the operator rotates and pushes the gripping part to fixate the fixing part into a uterine myoma or controls the gripping part to move the position of the uterine myoma in all directions, thereby mitigating the force exerted on the tips of the operator's hand.

In accordance with an aspect of the present invention, there is provided an apparatus for firmly fixing a uterine myoma of a patient to extract the uterine myoma easily during a laparoscopic myomectomy, wherein the apparatus is introduced into an abdominal cavity of the patient through a cannula which penetrates the abdominal cavity of the patient, the apparatus including: a body part having a cross-sectional diameter smaller than that of the cannula; a fixing part, having a shape of a circular cone, united together with one end of the body part, for fixing the uterine myoma firmly, to thereby facilitate the extraction of the uterine myoma; and a gripping part, connected to the other end of the body part, for applying traction force to the fixing part fixed into the uterine myoma, to obtain a sufficient view of an operative field in a restricted space created by the cannula.

### BRIEF DESCRIPTION OF THE DRAWINGS

The above and other objects and features of the present invention will become apparent from the following description of preferred embodiments given in conjunction with the accompanying drawings, in which:

FIG. 1 shows a general view of a conventional myoma screw for fixing a uterine myoma in laparoscopic myomectomy; and

FIG. 2 represents an apparatus for fixing a uterine myoma in the laparoscopic myomectomy in accordance with the present invention.

### DETAILED DESCRIPTION OF THE PREFERRED EMBODIMENTS

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the invention, although different, are not necessarily mutually exclusive. For example, a particular feature, structure, or characteristic described herein in connection with one embodiment may be implemented within other embodiments without departing from the spirit and scope of the invention. In addition, it is to be understood that the location or arrangement of individual elements within each disclosed embodiment may be modified without departing from the spirit and scope of the invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

The present invention will now be described in more detail, with reference to the accompanying drawings.

FIG. 2 represents an apparatus 100 for firmly fixing a uterine myoma, e.g., a large-sized uterine myoma, enabling an operator to get rid of the uterine myoma with ease during a laparoscopic myomectomy in accordance with a preferred embodiment of the present invention.

Referring to FIG. 2, the apparatus 100 includes a body part 101, a fixing part 102 and a gripping part 103.

The body part 101, made of metal with a length of about 30 cm, has a cylindrical shape, whose cross-sectional diameter is about 1 cm. The body part 101 may be introduced into an abdominal cavity following the fixing part 102 through a cannula (not shown) during an operation.

The fixing part 102, made of metal with a length of about 4.1 cm, is integrated with one end of the body part 101. In detail, the fixing part 102 has a shape of a circular cone on which spiral grooves are provided. Owing to the characteristic shape, the fixing part 102 can be firmly and deeply fixated into the uterine myoma, such that the fixing part 102 may not be easily pulled out or broken, even when the operator exerts the traction force on the uterine myoma by controlling the gripping part 103 for a better view of an operative field. Thus, a capsule of the uterine myoma may be efficiently dissected during the laparoscopic myomectomy.

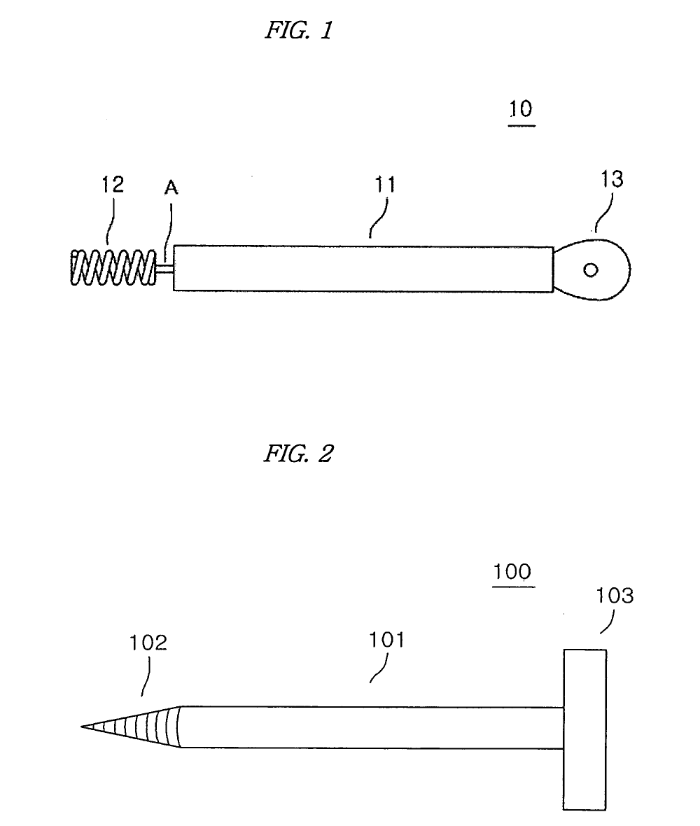

In particular, in case of a softened large-sized uterine myoma, with a diameter of 8 cm or more, having undergone the degenerative change, it is very difficult to firmly fix the uterine myoma by using the conventional myoma screw 10 as shown in FIG. 1.

However, the fixing part 102 can be deeply inserted and firmly fixated into the softened large-sized uterine myoma, so that the better view of the operative field can be obtained during the laparoscopic myomectomy by varying the position of the uterine myoma with the aid of the fixing part 102. Therefore, the softened large-sized uterine myoma can be fully extracted from the normal uterine tissue, while preventing the joint between the fixing part 102 and body part 101 from being broken.

The gripping part 103 has a cylindrical shape with a diameter of about 1.2 cm and a length of about 12 cm. In addition, the gripping part 103 is connected to the other end of the body part 101 by way of, e.g., an argon welding.

The uterine myoma can be pulled or pushed by controlling the gripping part 103 in a wanted direction, thereby obtaining a sufficient view of the operative field.

In particular, it is possible for the operator to turn the gripping part 103 by using his or her entire palm when the fixing part 102 is required to be inserted into the uterine myoma. Accordingly, the force of the operator can be exactly and efficiently delivered to the fixing part 102.

Besides, the force exerted on the gripping part 103 in a specific direction may be easily delivered to the fixing part 102 owing to the leverage effect, because the integrated shape of the gripping part 103 and the body part 101 is similar to the shape of ‘T’. In detail, a longitudinal direction of the gripping part 103 is perpendicular to a longitudinal direction of the body part 101 and the gripping part 103 is symmetrical if viewed from a central longitudinal axis of the body part 101. Further, the gripping part 103 has a cylindrical shape, whose height is parallel with the longitudinal direction of the gripping part 103.

Therefore, even in case the operator is worn to a frazzle by hard operation, the possibility of suffering from the arthralgia at the operator′ hand is greatly decreased. In addition, the shape of ‘T’ allows the gripping part 103 to function as a supporter capable- of supporting the fixing part 102 and the body part 101 so as not to be moved at the upper side of the abdomen, thereby facilitating the safe and rapid extraction of the uterine myoma.

Hereinafter, surgical procedures performed by using the apparatus 100 in accordance with the present invention will be described in detail, which are set forth to illustrate, but are not to be construed to limit the present invention.

A subpopulation of 36 patients whose myomas with sizes of 8 cm or more had been removed was selected from 103 patients who had underwent the laparoscopic myomectomy at Kangbuk Samsung Hospital from July 2003 to June 2006. All patients underwent preoperative assessments including detailed medical history, pelvic examination, gynecologic ultrasonography and Pap smear. The clinical charts, the operative and anesthetic records, and data on the patient's age, parity, operating time, diameter of the largest myoma, number of the removed myomas, hospital stay, change of the hemoglobin concentration from a day before the surgery to the postoperative day 1, operative indications, previous operative history, histopathological reports, complications and the like were reviewed. The total operating time was defined as the period from a starting time when a first trocar for introducing CO₂ into the abdominal cavity was inserted to an ending time when the port-site was seamed. The surgical procedure was performed under general anesthesia with endotracheal intubation. With the patient in a dorsal lithotomy position, a Foley catheter was inserted into the bladder to provide continuous urinary drainage before a uterine manipulator was fixed onto the cervix to perform a uterine manipulation.

In general, the surgical procedure for the laparoscopic myomectomy may include three steps as follows:

First, the large-sized uterine myoma is disscted from the normal uterine tissue while obtaining the safe and effective surgical vision. Second, the bleeding at the defect area of a uterine myometrium is controlled, and the defect area of the uterine myometrium is meticulously sutured to reconstruct it. Third, the resected uterine myoma is extracted from the abdominal cavity.

In specific, the first step functions as a step for obtaining the good surgical vision. In case of the uterine myoma whose size is large enough to reach the umbilical level, it was suggested that the trocar be located at the upper side of the umbilicus or an ancillary trocar be located at the upper abdomen. In the operations performed by using the apparatus 100 in accordance with the present invention, the trocar with a diameter of 5 mm was directly inserted into the infraumbilicus with a vertical skin incision without using a Veress needle. Carbon dioxide was insufflated through the trocar sleeve into the abdomen cavity to thereby create a pneumoperitoneum, and the intra-abdominal pressure was maintained at 15 mmHg. For patients with a very large uterus, at 16-18 gestational weeks, Choi's 4-trocar method was applied. In the upper side of a symphysis pubis, however, a trocar with a diameter of 12 mm was applied to use a morcellator and the apparatus 100 in accordance with the present invention.

Thereafter, a solution of vasopressin (Vasopressin®, Hanlim Pharm., Yongin, Korea) with an amount of 30 mL diluted with normal saline with a concentration of 10 IU/100 mL was injected into the tissue around the base and the capsule of the uterine myoma. Accordingly, the phenomenon of the vasoconstriction occurs, resulting in diminishing the amount of blood loss and obtaining the good surgical field, thus capable of discerning the uterine myoma from the normal uterine tissue.

Thereafter, a vertical incision was made in the most prominent portion of the uterine myoma in order to partially remove the capsule of the uterine myoma. Subsequently, the fixing part 102 and the body part 101 of the apparatus 100 were inserted into the most prominent part of the uterine myoma through the suprapubic trocar with a diameter of 12 mm, thereby fixing the uterine myoma firmly. Then, the operator held the gripping part 103 of the apparatus 100 with the entire palm of his left hand, and at the same time held metzembaum scissors or grasping forceps with his right hand, in order to dissect the uterine myoma. The first assistant, who held a telescope for laparoscope with a size of 5 mm in his left hand and grasping forceps in his right hand, helped the operator dissect the uterine myoma efficiently.

The fixing part 102 of the apparatus 100 fixated into the uterine myoma has a shape of a circular cone, which is united together with the body part 101 with a base plane of the circular cone, i.e., the fixing part 102, having the same diameter as that of the body part 101. Therefore, the joint between the fixing part 102 and the body part 101 may not be easily broken even if the traction force is exerted on the joint, thereby facilitating the extraction of the uterine myoma and the shortening the operative time.

In particular, in case the operative field in a uterus is restricted and the surgical instruments is difficult to control due to the large-sized uterine myoma, the view of the operative field could be obtained by moving-the gripping part 103 more extensively.

Besides, the larger the size of the uterine myoma or the uterus becomes, the more space could be obtained by moving the locations of the trocars, placed in both sides of the upper portion of the umbilicus, upward.

In addition, since the gripping part 103 can be easily controlled by using the entire palm of the operator's hand, while preventing excessive load from being delivered to some specific portions of the operator's fingers, the uterine myoma can be easily dissected to be located safely within a cul-de-sac by exerting the traction and countertraction force thereon efficiently.

At the above-mentioned second step, the defected area in the uterine myometrium was reconstructed and the effective hemostasis thereof was performed. The suture of a uterine wall using a bipolar coagulation or other effective hemostatic technique would lower the risk of bleeding. The uterine endometrium was examined to evaluate whether it was damaged. The approximation and the hemostasis were achieved in an inner myometrial layers by using interrupted sutures of 1-0 polyglactin 910 (Vicryl®, Ethicon Inc., Somerville, N.J.) by the help of an intracorporeal suture technique. However, the approximation was done in an outer myometrial layers and a serosa, by using the interrupted sutures with polyglycolic acid sutures (Lap-suture®, Sejong Medical, Seoul, Korea) by the help of an extracoporeal suture technique. Then, by suturing two or three times, the myometrial defect could be reconstructed and the strength of the uterine wall during a pregnancy period could be maintained. Moreover, by adjusting a myometrial edge accurately, the endometrial gland could be prevented from being proliferated in the uterine scar.

In case the size of defect area, after the surgery, for getting rid of a large uterine myoma is large, the extracorporeal suture technique would be more effective for the approximation, the hemostasis and the like. Besides, in order to diminish the amount of blood loss, oxytocin would be infused into patients during the surgery only after anesthesia or bilateral uterine artery ligation was adopted. In detail, the bilateral uterine artery ligation was selectively adopted for patients who wanted their uterus to be conserved although they did not want to be pregnant henceforth.

To use Gonadotropin-releasing hormone (GnRH) agonists prior to the surgery may be another choice, capable of significantly reducing the size of a uterine myoma; lowering its consistency; making the morcellation easier; and correcting the severe anemia. However, the uterine myoma may be too softened to handle; the surgical time may be prolonged because it is difficult to find the cleavage plane; the diagnosis of leiomyosarcoma may be delayed; and the small-sized uterine myoma may be shrunk to make the uterine myoma undetectable.

At the above-mentioned third step, the resected uterine myoma may be safely extracted from the abdominal cavity. To this end, the posterior colpotomy was adopted to pick out the resected uterine myoma through either vaginal or abdominal route using the morcellator, wherein the posterior colpotomy through the abdominal route was selected for most of the patients.

After the uterus was stitched, it was examined to determine whether uterine bleeding was present. If further bleeding was absent, the trocar with a diameter of 12 mm was removed. Then, the morcellator (X-tract Tissue Morcellator®, Gynecare, Somerville, N.J.) was introduced into the abdominal cavity to safely remove the uterine-myoma from the abdominal cavity. If there is not any notable bleeding detected, a meticulous examination was applied to search the possible residual tissue of the uterine myoma in the abdominal cavity. After intraabdominal irrigation was performed, the incision site was covered with an absorbable adhesion barrier (Interceed®, Gynecare, Somerville, N.J.) in order to prevent the adhesion. A drainage tube was inserted through the trocar with a diameter of 5 mm to extract the gas in the abdominal cavity.

All statistical analyses were performed using SAS program (V 9.1, SAS Institute Inc., NC). The patients' median age was 34 years (range 25-48 years), and the median parity was 0 (range 0-3). 6 patients (16%) had a previous operative history, including a cesarean section (3 patients), a laparoscopic tubal ligation (1 patient), and an appendectomy (2 patients). The most common operative indications were palpable abdominal mass (13 patients, 36%), pelvic pain (10 patients, 27%), abnormal uterine bleeding (9 patients, 25%), and urinary frequency (4 patients, 11%). Histopathological diagnosis included 34 cases of leiomyoma (94.4%), 2 cases of leiomyoma with adenomyosis (5.6%). The median diameter of the myomas was 8.6 cm (range 8-15.2 cm). In two cases, the diameters of the myoma were greater than or equal to 15 cm (e.g., 15.2 cm and 15.0 cm). In six cases, the diameters of the myoma were included in the range between 10 cm and 14 cm. The total number of the resected myomas was 76. In regard to the location of the uterine myoma, it was classified as an intramural type in 32 cases and a subserosal type in 4 cases. The maximum weight of the resected uterine myoma was 550 gm. The median operating time was 85 minutes (range 35-210 minutes). The median change in hemoglobin concentration from a day prior to the surgery to postoperative day 1 was 1.9 g/dL (range 0.1-4.7 g/dL). The median hospital stay was 3 days (range 2-6 days) Combined operations done with the laparoscopic myomectomy included a laparoscopic incidental appendectomy in 14 cases, an ovarian cystecystectomy in 4 cases, and an adhesiolysis in 3 cases. Of patients who did not want more pregnancy, in whom the severe bleeding was predicted, two patients underwent laparoscopic bilateral uterine artery ligation. The morcellator was used to extract the resected myomas in 33 patients. The posterior culdotomy was adopted to safely extract a myoma in 3 remaining patients. None of the procedures had been converted to laparotomy. There was no notable side effects during the surgery. 6 patients underwent transfusion intra-operatively or post-operatively, of whom 5 received 2 pints of packed red blood cell (RBC) and one received 4 pints thereof. A subcutaneous emphysema occurred in one patient, but no further events were noted after a conservative treatment. 3 patients became pregnant after the laparoscopic myomectomy, of whom one patient gave birth to a healthy baby by cesarean section due to a breech presentation. No intra-abdominal adhesion was noted during the surgery. 3 patients are pregnant at the present.

As described above, a number of the patients were recovered with no specific complications after the rapid and safe extraction of the uterine myomas owing to such an apparatus 100 in accordance with the present invention. Therefore, the aforementioned considerable operations performed by using the apparatus 100 have established the excellence of the apparatus 100 in the laparoscopic myomectomy.

While the invention has been shown and described with respect to the preferred embodiments, it will be understood by those skilled in the art that various changes and modifications may be made without departing from the spirit and the scope of the invention as defined in the following claims.

What is claimed is:

1. An apparatus for firmly fixing a uterine myoma of a patient to extract the uterine myoma easily during a laparoscopic myomectomy, wherein the apparatus is introduced into an abdominal cavity of the patient through a cannula which penetrates the abdominal cavity of the patient, the apparatus comprising:
a body part having a cross-sectional diameter smaller than that of the cannula;
a fixing part, having a shape of a circular cone, united together with one end of the body part, for fixing the uterine myoma firmly, to thereby facilitate the extraction of the uterine myoma; and
a gripping part, connected to the other end of the body part, for applying traction force to the fixing part fixed into the uterine myoma, to obtain a sufficient view of an operative field in a restricted space created by the cannula.

2. The apparatus of claim 1, wherein spiral grooves are provided on a conic surface of the fixing part.

3. The apparatus of claim 1, wherein the gripping part is rotated and pushed to insert the fixing part into the uterine myoma.

4. The apparatus of claim 3, wherein the shape of the integration between the gripping part and the body part is similar to the shape of ‘T’, enabling an operator to rotate and push the gripping part by using his or her entire palm.

5. The apparatus of claim 4, wherein a longitudinal direction of the gripping part is perpendicular to a longitudinal direction of the body part.

6. The apparatus of claim 5, wherein the gripping part has a cylindrical shape, whose height is parallel with the longitudinal direction of the gripping part.

7. The apparatus of claim 6, wherein the traction force applied to the gripping part is transferred to the fixing part by the leverage effect, thus obtaining the sufficient view of the operative field.

8. The apparatus of claim 7, wherein the gripping part is symmetrical if viewed from a central longitudinal axis of the body part.

9. The apparatus of claim 8, wherein the traction force is exerted onto the uterine myoma by moving the position of the gripping part.

10. The apparatus of claim 9, wherein the gripping part functions as a supporter for supporting the body part and the fixing part in the upper side of the abdomen.

11. The apparatus of claim. 10, wherein the gripping part is connected to the body part by way of welding.

12. The apparatus of claim 1, wherein a shape of a cross section of the body part is similar to that of the fixing part at an interface therebetween.

13. The apparatus of claim 1, wherein the fixing part is a metallic object.

14. The apparatus of claim 13, wherein the fixing part is about 4.1 cm in length.

15. The apparatus of claim 13, wherein the fixing part is about 1 cm in diameter.

16. The apparatus of claim 1, wherein the body part is a metallic object.

17. The apparatus of claim 15, wherein the body part is about 30 cm in length.

18. The apparatus of claim 15, wherein the body part is about 1 cm in diameter.

19. The apparatus of claim 1, wherein the gripping part is a metallic object.

20. The apparatus of claim 19, wherein the gripping part is about 12 cm in length.

21. The apparatus of claim 19, wherein the gripping part is about 1.2 cm in diameter.
